(19) United States
(12) Patent Application Publication
(10) Pub. No.: US 2008/0254073 A1
(43) Pub. Date: Oct. 16, 2008

(54) Transdermal Patch Comprising Paroxetine

(75) Inventor: Sang Cheol Chi, Gyeonggi-do (KR)

Correspondence Address: LEXYOUME IP GROUP, LLC, 1233 TWENTIETH STREET, N.W., SUITE 701, WASHINGTON, DC, 20036, US

(73) Assignee: IKSU PARMACEUTICAL CO., LTD., GWANGJU-SI (KR)

(21) Appl. No.: 11/995,488

(22) PCT Filed: Jul. 11, 2006
(86) PCT No.: PCT/KR06/02721

§ 371 Date: Jan. 11, 2008

(30) Foreign Application Priority Data
Jul. 22, 2005 (KR) 10-2005-0066877

(51) Int. Cl. A61K 9/00; A61K 31/4525; A61P 25/24

(52) U.S. Cl. 424/400; 514/321

(57) ABSTRACT

Disclosed relates to a transdermal patch comprising paroxetine that is useful to reduce the side effects accompanied with the initial high drug concentration after oral administration of paroxetine and decrease the broad metabolism of the drug in the liver. Moreover, the transdermal patch comprising paroxetine of the present invention has an excellent skin permeation rate and shows high bioavailability compared with oral administration of the drug.

### TECHNICAL FIELD

The present invention relates to a transdermal patch comprising paroxetine.

### BACKGROUND ART

Up to now, it has been known that the depressive disease is caused by an inappropriate secretion of serotonin, known as a major hormone that aids biorhythm regulation together with melatonin. Serotonin is a neurotransmitter secreted in the brain and has an emotion-regulating function. An unequal concentration of serotonin may induce diseases such as a depression. Accordingly, researchers have paid attention to the therapeutics that maintains the concentration of serotonin. Such therapeutics is to increase the amount of serotonin by administrating a selective serotonin reuptake inhibitor (hereinafter, referred to as SSRI) to a patient to inhibit the neuroterminal from taking serotonin again, a communication means of cranial nerves and other nerves, thus treating the depression. Paroxetine of the present invention, one of the SSRIs providing such function, has been known as an effective agent for preventing and treating various diseases such as depression, compulsive disease, panic disorder, obesity, senile dementia, migraine, etc.

Paroxetine is a viscous oil of weak yellow, which is very slightly soluble in water (0.26 [symbol] ), however, has excellent miscibility with various solvents. The partition coefficient of the drug using octanol-water is 169.20±17.19 (log P=2.23).

Since paroxetine is a viscous liquid that is very slightly soluble in water, it is not easy to handle. Accordingly, paroxetine is converted to solid salt forms which are easy to handle and suitable for oral administration.

Paroxetine has been used in the form of acidic salt, particularly, hydrochloride having excellent biocompatibility. However, crystalline paroxetine hydrochloride has low bioavailability and, accordingly, shows considerable fluctuation in the plasma concentration of the drug when administered to a patient, since it has low water solubility (6 to 12 [symbol] ). Compared with this, amorphous paroxetine hydrochloride has high water solubility (75 [symbol] ), however, it has high hygroscopicity, bad stability and bad flowability which makes it difficult to handle. To solve this problem, U.S. Pat. No. 6,168,805 has disclosed a process for preparing solid, amorphous paroxetine hydrochloride by mixing the drug with polymer and drying them to make solid dispersion. The result product has good handling properties. In addition, an invention related to crystalline paroxetine hydrochloride hemihydrate has been disclosed in U.S. Pat. No. 4,721,723, wherein such crystalline substance has good flowability and, thus, good handling properties. However, its preparation process is more complicated than the amorphous compositions.

Paroxetine has been administered via oral route and its usual dose is 20 to 40 [symbol] . Reviewing its pharmacokinetic properties after oral administration of the drug, the time to reach maximum plasma concentration of the drug is about five hours after its oral administration to healthy volunteers and its range is considerably wide as 0.5 to 11 hours. Moreover, the deviation of the maximum plasma concentration of the drug is shown very large as 0.8 to 32.5 ng/ [symbol] after the administration of 20 [symbol] and 2.5 to 65.1 ng/ [symbol] after administration of 50 [symbol] . It is due to significant differences among individuals in presystemic metabolism. There is a difference about 35 times in the area under the plasma concentration-time curve (AUC). The elimination half-life of paroxetine is about 24 hours after multiple oral administration of the drug to healthy volunteers and its range is considerably wide as 3 to 65 hours. Paroxetine is extensively metabolized in the liver and about 1 to 2% of the administered amount is excreted as unchanged drug via the urine.

Paroxetine shows various side effects including nausea like other SSRIs (i.e., fluoxetine, sertraline, citalopram, fluvoxamine, etc.). It has been known that such side effects relate to the initial high concentration of the drug appeared after its oral administration. In the past, various methods aimed at reducing such side effects were attempted.

For example, there has been a method that reduces the side effects by decreasing the dose, which is advantageous in view of the reduction in the side effects, however, disadvantageous in view of the therapeutic value, since the plasma concentration decreases, too.

Another method aimed at delaying the absorption rate of the drug physically by administration of food or antacid together with paroxetine has been attempted. However, there is a drawback in that the absorption of paroxetine is not influenced by food or antacid.

In addition, another method of administering paroxetine in the sustained-release dosage form has been attempted. As a result, it was shown that paroxetine of the sustained-release dosage form lowered the plasma concentration of the drug at early stage compared with an immediately release dosage form, thus, reducing the frequency of side effects. That is, a study on pharmacokinetic properties of the sustained-release dosage form after oral administration has shown that it is possible to delay the time to reach the maximum plasma concentration of the drug for 4 to 5 hours compared with the immediately release dosage form, thus lowering the maximum plasma concentration of the drug and maintaining the drug concentration in plasma relatively high during the elimination phase.

Accordingly, the inventor of the present invention has examined various available methods aimed at reducing the side effects of paroxetine accompanied with the initial high concentration of paroxetine after its oral administration and decreasing the extensive metabolism proceeding in the liver and found that it is the most appropriate method to formulate paroxetine as a transdermal patch, thus completing the present invention that provides a transdermal patch comprising paroxetine and having excellent skin permeation rate and high bioavailability than its oral administration.

### DISCLOSURE OF INVENTION

### Technical Problem

An object of the present invention is to provide a transdermal patch comprising paroxetine that reduces side effects accompanied with oral administration of the drug and has high bioavailability.

### Technical Solution

To accomplish the object of the present invention, there is provided a transdermal patch comprising paroxetine having excellent skin permeation rate.

### ADVANTAGEOUS EFFECTS

Accordingly, it can be understood that the transdermal patch comprising paroxetine of the present invention is useful to reduce the side effects accompanied with the initial high drug concentration after oral administration of paroxetine and decrease the broad metabolism of the drug in the liver. Moreover, the transdermal patch comprising paroxetine of the present invention has excellent skin permeation rate of the drug and shows high bioavailability compared with its oral administration, thus being expected that it can be substituted for the conventional administration route of paroxetine.

### BRIEF DESCRIPTION OF THE DRAWINGS

FIG. 1 is a graph showing a skin permeation profile of paroxetine in accordance with Experimental Example 1 of the present invention; and

FIG. 2 is a graph depicting a plasma concentration-time profile of paroxetine in accordance with Experimental Example 2 of the present invention (◯: Variation of plasma concentration after transdermal application of paroxetine patch, : Variation of plasma concentration after oral administration of paroxetine tablet).

### BEST MODE FOR CARRYING OUT THE INVENTION

A transdermal patch comprising paroxetine in accordance with the present invention is a matrix type patch comprising: (1) a backing film; (2) a drug-contained adhesive layer; and (3) a release liner.

In the transdermal patch comprising paroxetine of the present invention, the backing film is designed to be thin and soft and does not have reactivity with skin which may induce skin allergy. As the backing film, it is possible to use a single layer, such as polyester, polyurethane, polyethylene, polypropylene, polyolefin, polyethylene terephthalate, polyester with aluminum, etc., or a multilayered laminate film combining such single layer with nonwoven fabric, cotton fabric, woven fabric, etc., which have water absorption for preventing the patch from being removed due to moisture coming out from the skin. Moreover, any kind of drug protecting films used in the conventional patches may be adopted.

In the transdermal patch comprising paroxetine of the present invention, the drug-contained adhesive layer may include any kinds of pressure sensitive adhesives without limitations and, preferably, the pressure sensitive adhesive is made of acrylate-based polymer having a hydroxyl group, acrylate-based polymer having no functional group, acrylate-vinylacetate-based polymer having a hydroxyl group or acrylate-vinylacetate-based polymer having no functional group.

In the transdermal patch comprising paroxetine of the present invention, the drug-contained adhesive layer may use at least one selected from the group consisting of isopropyl myristate, Transcutol, triacetin, pyrrolidone derivatives, fatty acids, fatty acid alcohols and esters, as a general vehicle, a skin penetration enhancer, or an additive. In according with the present invention, it is desirable that the fatty acid alcohol is dodecyl alcohol and the pyrrolidone derivative is N-methylpyrrolidone.

In the transdermal patch comprising paroxetine of the present invention, it is desirable to regulate the content of the general vehicle, the skin penetration enhancer or the additive to be 1˜20% by weight for the weight of the drug-contained adhesive layer. If the content of the general vehicle, the skin penetration enhancer or the additive is less than 1% by weight, it has no effect on improving the skin permeation rate of the drug. Moreover, if the content exceeds 20% by weight, the skin permeation of the drug is no longer improved; further, it may deteriorate the physical strength of the patch.

In the transdermal patch comprising paroxetine of the present invention, it is desirable that the content of paroxetine contained in the drug-contained adhesive layer is 5˜20% by weight for the weight of the drug-contained adhesive layer. If the content of paroxetine is less than 5% by weight, it is difficult to reach a sufficient, effective blood concentration of the drug. On the contrary, if the content of paroxetine exceeds 20% by weight, it causes drug crystallization in the product and the skin permeation of the drug is not increased further.

Although paroxetine has good properties as the candidate of a transdermal preparation, a patch fabricated with paroxetine itself has low skin permeation rate. For example, when measuring the skin permeation rate using excised rat skins for 30 hours with a paroxetine patch comprising an acrylate-based adhesive containing a carboxyl group as a functional group or an acrylate-vinylacetate-based adhesive, no skin permeation occurred. Moreover, in case of a paroxetine patch comprising the same adhesive containing a hydroxyl group, the skin permeation rate of the drug was only 2.3 to 4.5 [symbol] /hr.

Meanwhile, a paroxetine patch having a thickness of 100 [symbol] prepared in accordance with a preferred embodiment of the present invention resulted in high skin permeation rate of 39.3 [symbol] /hr. That is, the transdermal patch added with the general vehicle, the skin penetration enhancer or the additive in accordance with the present invention can show excellent skin permeation of paroxetine corresponding to 100 to 3,000 [symbol] /hr with appropriate application area.

In the transdermal patch comprising paroxetine of the present invention, the release liner plays a role of supporting the product when cutting the patches in appropriate sizes and is to be removed before applying the product to the skin. It is possible to apply a film such as aluminum, cellulose, polyester, polyethylene and polypropylene or a thin membrane made of paper and to laminate such films if necessary. Moreover, it is desirable that the release liner be readily removed from the patch, not leaving matrix remains on the release liner and, further, any kind of materials or forms that have been applied generally to the transdermal patches may be used.

A preparing method of the transdermal patch comprising paroxetine of the present invention will now be described as follows.

Paroxetine and necessary additives are added to the solution containing a pressure sensitive adhesive and dissolved under stirring, thus preparing a homogeneous drug-contained adhesive solution. The adhesive solution is spread over a release liner using an appropriate equipment and dried. The dried liner is laminated to a backing film to prepare a paroxetine patch comprising a drug-contained adhesive layer.

In the transdermal patch comprising paroxetine of the present invention, the thickness of the drug-contained adhesive layer is desirably within a range of 50 to 300 [symbol] and the thickness of the adhesive layer may be varied freely. Moreover, it is desirable that the transdermal patch comprising paroxetine of the present invention be applied to the skin one time over a period of one to three days and the area applied to the skin once be 2.5˜70 [symbol] .

### MODE FOR THE INVENTION

Hereinafter, the present invention will now be described more fully with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

### EXAMPLE 1

### Preparing of Transdermal Paroxetine Patch of the Present Invention I

As an adhesive, Duro-Tak 87-9301, a solution containing an acrylate-based polymer having no functional group (supplied by the National Starch & Chemical Company, USA) was used.

To 77.5 g of Duro-Tak 87-9301 as solid content, 10 g of paroxetine, 6 g of isopropyl myristate, 5 g of triacetin and 1.5 g of Transcutol (supplied by Gattefosse SA, France) were added and dissolved under stirring, thus preparing a homogeneous drug-contained adhesive solution. The prepared adhesive solution was spread over a polyester release liner (3M Scotchpak 1022, USA) using a labcoater (supplied by Mathis AG, Switzerland) and dried using a labdryer (supplied by Mathis AG, Switzerland) at 70° C. for one hour. The dried liner was laminated to a polyester backing film (3M Scotchpak 9732, USA), thus preparing a transdermal paroxetine patch comprising a drug-contained adhesive layer having a thickness of 60 [symbol] (paroxetine content: 10% by weight).

### EXAMPLE 2

### Preparing of Transdermal Paroxetine Patch of the Present Invention II

As an adhesive, Duro-Tak 87-9301, a solution containing an acrylate-based polymer having a hydroxyl group (supplied by the National Starch & Chemical Company, USA) was used.

To 67.5 g of Duro-Tak 87-9301 as solid content, 20 g of paroxetine, 6 g of isopropyl myristate, 5 g of dodecyl alcohol and 1.5 g of Transcutol were added and dissolved under stirring, thus preparing a homogeneous drug-contained adhesive solution. The prepared adhesive solution was spread over a polyester release liner (3M Scotchpak 1022, USA) using a labcoater (supplied by Mathis AG, Switzerland) and dried using a labdryer (supplied by Mathis AG, Switzerland) at 70° C. for one hour. The dried liner was laminated to a polyester backing film (3M Scotchpak 9732, USA), thus preparing a transdermal paroxetine patch comprising a drug-contained adhesive layer having a thickness of 150 [symbol] (paroxetine content: 20% by weight).

### EXAMPLE 3

### Preparing of Transdermal Paroxetine Patch of the Present Invention III

As an adhesive, Duro-Tak 87-4098, a solution containing an acrylate-vinylacetate-based polymer having no functional group (supplied by the National Starch & Chemical Company, USA) was used.

To 75 g of Duro-Tak 87-4098 as solid content, 5 g of paroxetine and 20 g of N-methylpyrrolidone were added and dissolved under stirring, thus preparing a homogeneous drug-contained adhesive solution. The prepared adhesive solution was spread over a polyester release liner (3M Scotchpak 1022, USA) using a labcoater (supplied by Mathis AG, Switzerland) and dried using a labdryer (supplied by Mathis AG, Switzerland) at 70° C. for one hour. The dried liner was laminated to a polyester backing film (3M Scotchpak 9732, USA), thus preparing a transdermal paroxetine patch comprising a drug-contained adhesive layer having a thickness of 250 [symbol] (paroxetine content: 5% by weight).

### EXPERIMENT EXAMPLE 1

### Measurement of Skin Permeation Rate of Paroxetine

The following experiment was carried out for determining the skin permeation rate of the drug from the transdermal paroxetine patch of the present invention.

Franz diffusion cells fitted with human cadaver skins were used for measuring the skin permeation rate of paroxetine from the transdermal patch prepared in Example 1. About 11.5 [symbol] of pH 7.4 phosphate buffer was used as a receptor solution and the skin permeation area was 1.77 [symbol] .

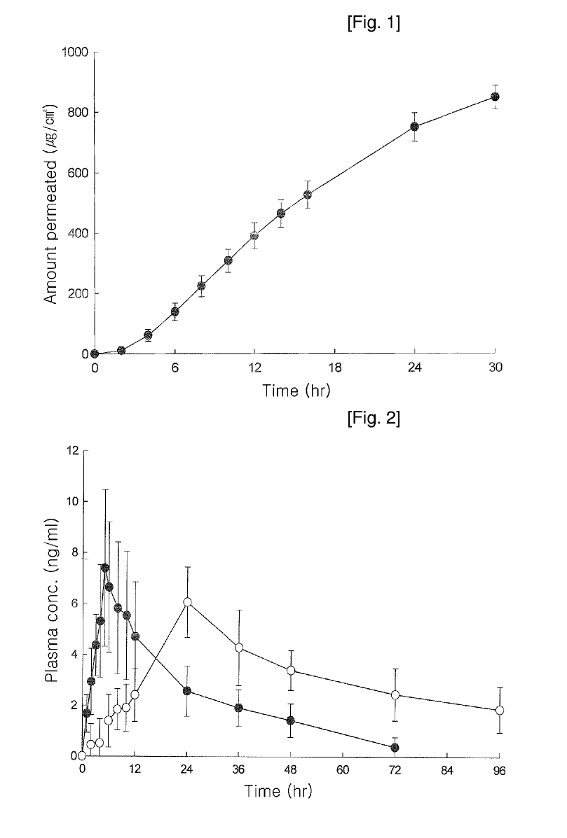

After the patch was applied to the skin, the receptor solution was collected for 10 hours at predetermined time intervals. The contents of paroxetine were quantitated using high performance liquid chromatography. The skin permeation profile of paroxetine from the patch was obtained and the skin permeation rate of the drug was calculated from a straight line of the profile after the lag time. The result was depicted in FIG. 1. From the result, it could be understood that the skin permeation rate (n=7) of paroxetine was shown as high as 39.54±2.56 [symbol] /hr.

### Experiment Example 2

### Pharmacokinetic Experiment

After transdermal application of the transdermal paroxetine patch prepared in Example 1 to healthy volunteers, pharmacokinetic properties were measured. Relative bioavailability was also calculated compared with oral administration of paroxetine.

Twelve health males aged 25 to 47 years and weighed 55 to 85 kg were randomly divided into two groups of six subjects. The transdermal paroxetine patches of the present invention were applied to one group and the paroxetine tablets (Seroxat Tab., 20 [symbol] of paroxetine contained in a tablet) were orally administrated to the other group. For the transdermal application group, the patches of 32 [symbol] size comprising 20 [symbol] of paroxetine were applied to forearms for 24 hours and removed.

After administrations of the two products, bloods were collected for 96 hours from the transdermal application group and for 72 hours from the oral administration group at predetermined time intervals. The concentration of paroxetine in the obtained plasma was quantitated using high performance liquid chromatography and the result was depicted in FIG. 2. The relative bioavailability of the paroxetine patch was 151%, from which it could be learned that the transdermal applications showed excellent absorption rate compared with the oral administration of the drug.

Although the present invention has been described with reference to certain exemplary embodiments thereof, it will be understood by those skilled in the art that a variety of modifications may be made therein without departing from the spirit or scope of the present invention defined by the appended claims and their equivalents.

1. A transdermal patch comprising paroxetine comprising: (1) a backing film; (2) a drug-contained adhesive layer including paroxetine; and (3) a release liner.

2. The transdermal patch comprising paroxetine as recited in claim 1,
wherein the drug-contained adhesive layer uses polymer as a pressure sensitive adhesive made of acrylate-based polymer having a hydroxyl group, acrylate-based polymer having no functional group, acrylate-vinylacetate-based polymer having a hydroxyl group or acrylate-vinylacetate-based polymer having no functional group.

3. The transdermal patch comprising paroxetine as recited in claim 1,
wherein the content of paroxetine in the drug-contained adhesive layer is 5˜20% by weight for the weight of the drug-contained adhesive layer.

4. The transdermal patch comprising paroxetine as recited in claim 1,
wherein the thickness of the drug-contained adhesive layer is 50 to 300 μm.

5. The transdermal patch comprising paroxetine as recited in claim 1,
wherein the drug-contained adhesive layer further comprises a conventional vehicle, a skin penetration enhancer, or an additive.

6. The transdermal patch comprising paroxetine as recited in claim 5,
wherein the content of the conventional vehicle, the skin penetration enhancer or the additive is 1˜20% by weight for the weight of the drug-contained adhesive layer.

7. The transdermal patch comprising paroxetine as recited in claim 5,
wherein the conventional vehicle, the skin penetration enhancer or the additive is at least one selected from the group consisting of isopropyl myristate, Transcutol, triacetin, pyrrolidone derivatives, fatty acids, fatty acid alcohols and esters.

8. The transdermal patch comprising paroxetine as recited in claim 7,
wherein the pyrrolidone derivative is N-methylpyrrolidone.

9. The transdermal patch comprising paroxetine as recited in claim 8,
wherein the fatty acid alcohol is dodecyl alcohol.

10. The transdermal patch comprising paroxetine as recited in claim 1 having a skin permeation rate of 100 to 3,000 μg/hr.

11. The transdermal patch comprising paroxetine as recited in claim 2 having a skin permeation rate of 100 to 3,000 μg/hr.

12. The transdermal patch comprising paroxetine as recited in claim 3 having a skin permeation rate of 100 to 3,000 μg/hr.

13. The transdermal patch comprising paroxetine as recited in claim 4 having a skin permeation rate of 100 to 3,000 μg/hr.

14. The transdermal patch comprising paroxetine as recited in claim 5 having a skin permeation rate of 100 to 3,000 μg/hr.

15. The transdermal patch comprising paroxetine as recited in claim 6 having a skin permeation rate of 100 to 3,000 μg/hr.

16. The transdermal patch comprising paroxetine as recited in claim 7 having a skin permeation rate of 100 to 3,000 μg/hr.

17. The transdermal patch comprising paroxetine as recited in claim 8 having a skin permeation rate of 100 to 3,000 μg/hr.

18. The transdermal patch comprising paroxetine as recited in claim 9 having a skin permeation rate of 100 to 3,000 μg/hr.
